(19) United States
(12) Patent Application Publication
(10) Pub. No.: US 2015/0201869 A1
(43) Pub. Date: Jul. 23, 2015

(54) HANDHELD VIBRATING MEDICAL DEVICE FOR SENSORY DIAGNOSTICS

(71) Applicant: Mauziar Nikzad, Bellevue, WA (US)

(72) Inventor: Mauziar Nikzad, Bellevue, WA (US)

(21) Appl. No.: 14/162,729

(22) Filed: Jan. 23, 2014

(51) Int. Cl. A61B 5/12; A61B 1/227

(52) U.S. Cl. CPC A61B 5/12; A61B 1/227

(57) ABSTRACT

A method and a device are disclosed for the diagnosis of sensory sensitivity in medical patients, including using a multifunctional medical device which may be utilized to test patient's skin sensitivity to a variety of stimuli and conditions such as heat or temperature, vibrations, electrical shock, pressure, pain, burning sensation, and the like. In some embodiments, the medical device includes a multifunction diagnostic head providing various stimuli at its tip, such as various temperatures, vibration, electrical shock, pressure, pain, and other stimuli which is detected by human skin sensory cells. In some embodiments, a diagnostic head coupled with a computing device may be used to generate sound for hearing test. In other embodiments, each of these stimuli may be provided by a separate removable diagnostic head. In other embodiments, a removable diagnostic head may provide several, but not all, or all of the desired sensory stimuli listed above.

### CROSS-REFERENCE(S) TO RELATED APPLICATION(S)

This application is related to the U.S. application Ser. No. 13/423,076, filed on 16 Mar. 2012, the disclosure of which is hereby expressly incorporated by reference in its entirety.

### TECHNICAL FIELD

This application relates generally to medical devices. More specifically, this application relates to a method and apparatus for assessing sensory perception.

### BRIEF DESCRIPTION OF THE DRAWINGS

The drawings, when considered in connection with the following description, are presented for the purpose of facilitating an understanding of the subject matter sought to be protected.

FIG. 1 shows an example otoscope with a removable diagnostic head;

FIG. 2 shows an example multifunctional medical diagnostic device with a removable diagnostic head;

FIG. 3 shows an example electronic thermo-cooling device;

FIG. 4A shows an example removable diagnostic head configured to test pressure sensation;

FIG. 4B shows an example removable diagnostic head configured to test pain sensation;

FIG. 4C shows an example removable diagnostic head configured to test touch discrimination;

FIG. 4D shows an example removable diagnostic head configured to test touch sensitivity;

FIG. 5 shows an example removable diagnostic head configured to test hearing acuity using a computing device;

FIG. 6 shows an example computing device similar to the computing device of FIG. 5;

FIG. 7A shows an example cone loudspeaker;

FIG. 7B shows an example graphene loudspeaker; and

FIG. 7C shows an example piezoelectric loud speaker.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description references a thermal sensitivity diagnostic head, it will be appreciated that the disclosure may be used with other types of sensory diagnostic heads such as pressure sensitivity, burning sensitivity, and the like.

Briefly described, a method and a device are disclosed for the diagnosis of sensory sensitivity in medical patients, including using a multifunctional medical device which may be utilized to test patient's skin sensitivity to a variety of stimuli and conditions such as heat or temperature, vibrations, electrical shock, pressure, pain, burning sensation, and the like. In some embodiments, the medical device includes a multifunction diagnostic head which can provide various stimuli at its tip, such as various temperatures, vibration, electrical shock, pressure, pain, and other stimuli which is detected by human skin sensory cells. In some embodiments, a hearing diagnostic head with loudspeaker coupled with a computing device may be used to generate a sound for hearing test. In other embodiments, each of these stimuli may be provided by a separate removable diagnostic head. In still other embodiments, a removable diagnostic head may provide several, but not all, or all of the desired sensory stimuli listed above. In further embodiments, a hearing test or diagnostic head may be used with the multifunctional medical device.

Many medical conditions or diseases have symptoms which are manifested on skin, particularly on finger tips or toe tips. Hence, such conditions or diseases may be diagnosed, at least partially, by assessing sensations a patient feels on his finger tips or toe tips as a result of measured or controlled stimuli applied to the finger tip. The utility of finger tip sensation tests are not limited to diagnosing diseases, but may be useful in a general health assessment, for example, during a physical exam.

Some of the conditions or diseases which may have sensory symptoms include diabetes, hypothyroidism, blood circulation problems, nervous system problems such as neuropathy (dying nerves), and the like.

Hearing Test

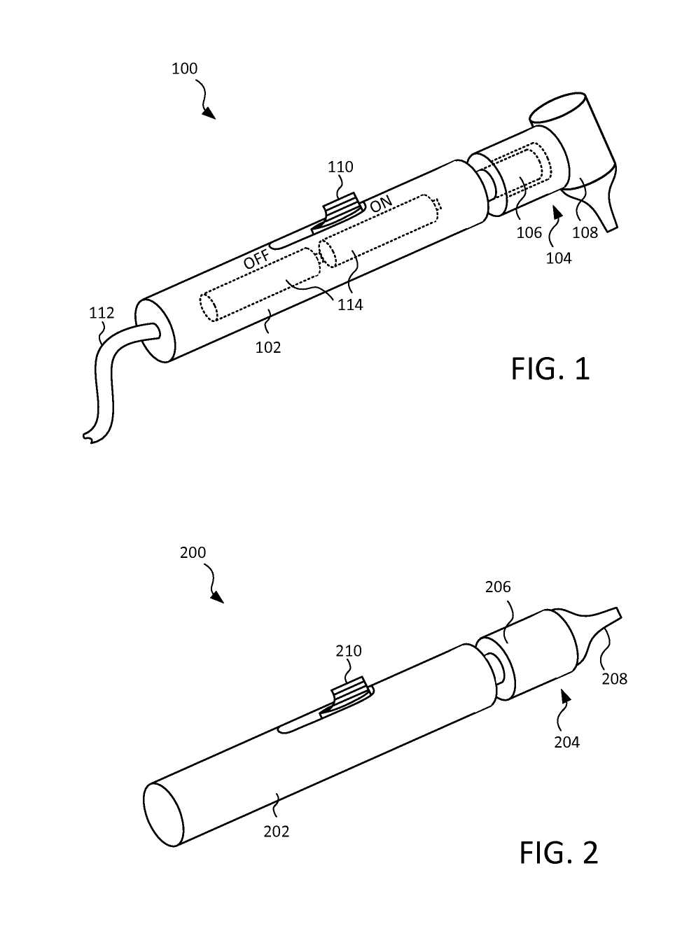

FIG. 1 shows an example otoscope with a removable diagnostic head. In some embodiments, otoscope 100 includes handle 102, ON/OFF switch 110, removable diagnostic head 104 including an attachment base 106, and diagnostic tip 108. Otoscope 100 may be powered, if needed, by batteries 114 or power cord 112.

Otoscope is typically used for diagnosing health problems of ear, nose, and throat by looking through a narrow, lighted, and sometimes magnified tip (using a lens) at the inside of ear, nose, or throat canals. In some embodiments, diagnostic head 104 may be removable to allow the attachment of the appropriate diagnostic tips with different shapes and functionalities. For example, a diagnostic tip for looking inside the ear may be different from one for looking inside the nose or throat.

Because Otoscope is a common hand-held medical device used in almost all physician's offices, it may be a good platform to use for other common diagnostic tests, such as testing various sensations on finger tips or toe tips. An otoscope provides a handle, a power source, and an interface for attaching different heads, which are all useful features for a general hand-held diagnostic device.

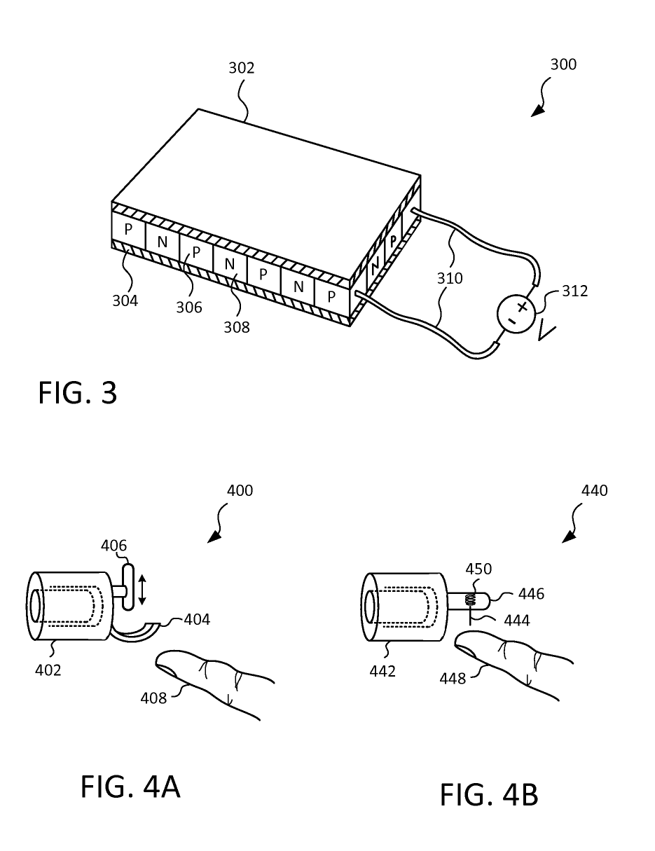

FIG. 2 shows an example multi functional medical diagnostic device with a removable diagnostic head. In some embodiments, medical diagnostic device 200 includes handle 202, removable diagnostic head 204 including attachment base 206 and diagnostic tip 208. Attachment base 206 is generally used to couple the diagnostic head to handle 202 of medical diagnostic device 200 using various coupling techniques, such as screw and threads, twist-lock, floating ball lock, and the like. In other embodiments, the medical diagnostic device may be a single integrated piece without a removable or separate diagnostic head. Those skilled in the art will appreciate that many other configurations of such medical device may be made without departing from the spirit of the present disclosures. For example, the handle may be similar to a gun-handle, rather than a straight one.

In various embodiments, diagnostic tip 208 may be configured to perform one or more testing functions including thermal (hot and cold), pressure, pain, electrical shock, burning, and other similar sensations.

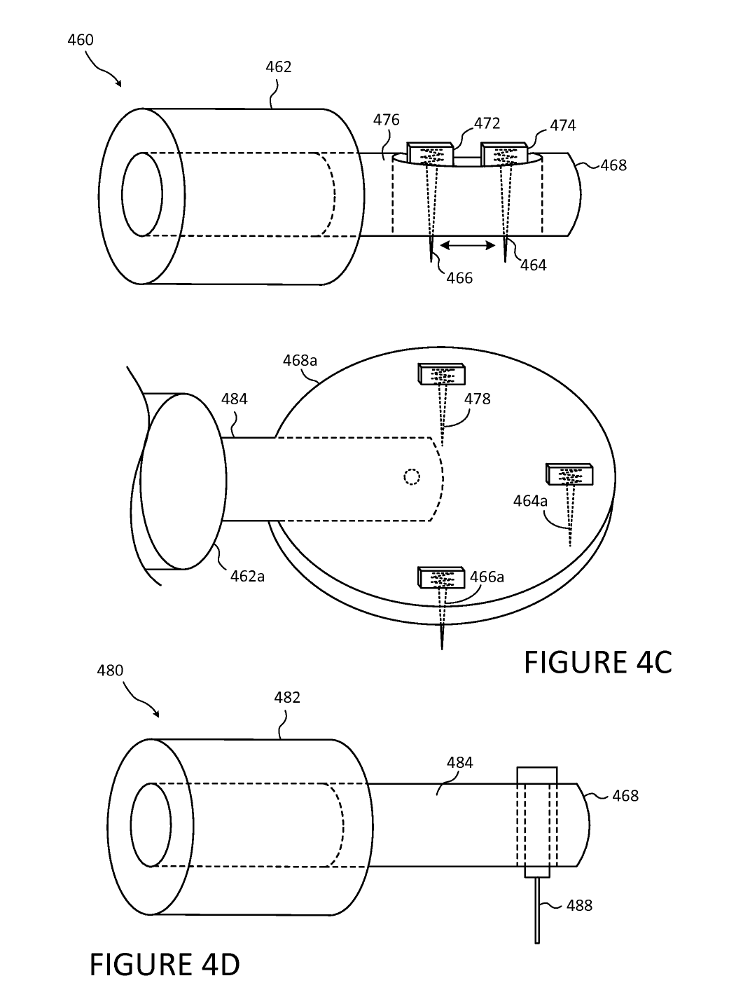

In various embodiments, diagnostic tip 208 is configured to provide thermal stimulus. In some embodiments, a preset temperature is provided by the tip upon turning on of the device. For example, different tips may be preset to different temperatures, hotter or colder than human body's temperature. In other embodiments, a temperature control slider or knob is provided to set the tip temperature to a desired value, using a built-in temperature control unit to regulate the temperature. The heat or cold may be provided using an thermoelectric heater and/or cooler, further described below with respect to FIG. 3. In some embodiments two or more thermoelectric coolers may be used for quick switching between hot and cold or for providing more heat capacity to heat or cool an area.

In application, diagnostic tip 208 is touched to the fingertip or other skin area of the patient to test the patient's sensory sensitivity to the particular stimulus, such as heat or cold. In various embodiments, the value or level of the stimulus may be recorded to be included in patient's records or consistently characterize the patient's sensitivity to the particular stimulus. For example, a patient may be sensitive to heat sensation only after the thermal stimulus is set to a value above 100° F., or cold sensation below 48° F. Similarly, other stimuli, such as vibration, pressure or electrical shock, may be set to particular values.

In some embodiments, diagnostic tip 208 may be coupled with a vibration generating component, such as a small electric motor, or armature. The shaft of the armature may be coupled to a cam in an off-center position to convert the rotational motion of the shaft to a vibratory motion. The vibration thus generated at the diagnostic tip may be used as a stimulus to test touch sensitivity. In other embodiments, a rotating brush or other similar articles may be used to stimulate the sense of touch.

In some embodiments, diagnostic tip 208 may be coupled to an electrical voltage and/or current source, such as a chargeable capacitor housed within the diagnostic head 204 or handle 202, to furnish, upon the activation of the diagnostic tip, a measured electrical charge to the patient's finger to test the patient's sensitivity to such stimuli. In some embodiments, the capacitor may be charged to a fixed and predetermined level, while in other embodiments, the charge of the capacitor may be programmable and/or adjustable.

FIG. 3 shows an example electronic thermo-cooling device. In various embodiments, electronic thermo-cooling device 300 includes a hot-side thermal surface 302 and a cold-side thermal surface 304 enclosing between them P-N junctions having positively doped, or P type, and negatively doped, or N type, semiconductors. Conductors 310 connected to Direct Current (DC) voltage source 312 cause a temperature differential to be established between the two thermal surfaces, making one surface hotter than the other and both surfaces hotter or colder than the ambient temperature. Such an arrangement essentially establishes a heat pump which pumps thermal energy from one conducting surface to the other, making the heat source surface colder and the heat target surface hotter.

In various embodiments, the thermo-cooling device is small enough that it may be built-in at or very near the diagnostic tip. This way, thermal efficiency is preserved and the desired temperature may be achieved more quickly without loss of heat.

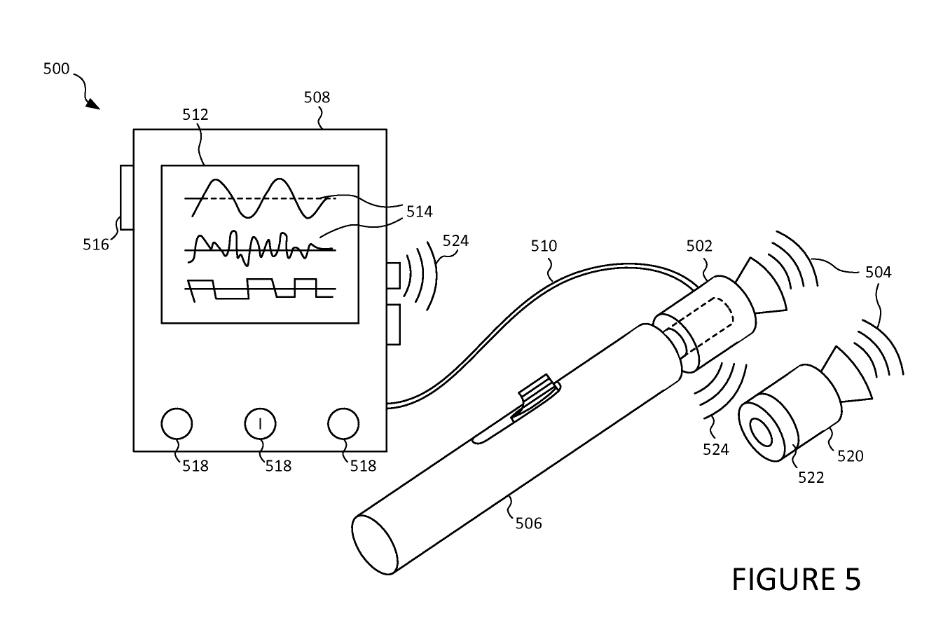

FIG. 4A shows an example removable diagnostic head configured to test pressure sensation. In various embodiments, removable diagnostic head 400 includes an attachment base 402, finger cradle 404, and pressure applicator component 406 used to apply a pressure to finger 408.

In various embodiments, pressure applicator component 406 is configured to move in a direction to exert a pressure on finger 408. For example, the pressure applicator may be a rod which can move up and down over the finger cradle to apply a measured pressure onto finger 408. A physician may then ascertain when the finger senses the pressure and whether its sensitivity is within normal medical range. In various embodiments, pressure head 400 may be preset to a particular fixed pressure so that once the pressure applicator is released or activated, it exerts a particular amount of force or pressure onto the finger. In other embodiments, pressure head 400 may include an adjustment control to variably set the exerted pressure to a desired level. Such adjustment may implemented using a spring tensioner arrangement, a wedge arrangement or other known mechanical techniques. The pressure adjustment mechanism may be calibrated to know what pressure it corresponds to.

Cradle 404 is used to support the finger and also to distribute the pressure evenly on the back of the finger (where fingernail is located) so the sensory focus of the patient is on the front of the finger facing pressure applicator 406.

FIG. 4B shows an example removable diagnostic head configured to test pain sensation. In various embodiments, diagnostic head 440 includes attachment base 442, diagnostic tip 446, pin 444 for lightly puncturing or pricking finger 448, and spring 450.

In various embodiments, pin 444 is coupled with spring 450 which, upon activation of diagnostic tip 446, lounges pin 444 towards finger 448 with a particular force and to a particular extent or distance to prick the finger in order to test pain sensitivity. In other embodiments, pin 444 may be driven using other techniques such as magnetic force, pneumatic force (like an air cylinder and piston arrangement), or other suitable techniques for driving the pin. In some embodiments, the force and extension distance of the pin towards the finger are predetermined, while in other embodiments, they are adjustable.

FIG. 4C shows an example removable diagnostic head configured to test touch discrimination. In various embodiments, touch discrimination diagnostic head 460 includes attachment base 462, slotted substrate 468 having slot 476 within which pins 464 and 466 are deployed coupled with base springs 472 and 474, respectively. In some embodiments, a planar substrate 468a may be used to support multiple pins 464a, 466a, and 478 distributed over a two-dimensional area and coupled with attachment base 462a.

In various embodiments, two or more pins with fixed or adjustable distance from each other may be used to test touch discrimination and resolution to assess whether a patient can tell the difference between two (or more) touch points on his skin, for example, hand or forearm, from a single touch point. This type of diagnostic may be use by surgeons, neurologists, other health care experts, or by patients themselves to assess status of neurological health.

In various embodiments, spring-loaded pins may be used to exert a predetermined amount of force on skin surface to test touch sensation and/or diagnose health problems. In some embodiments, the distance between the two (or more) pins are predetermined and fixed, while in other embodiments, the distance between the two pins are adjustable by the user. For example, the pins may be housed in a slot in the substrate and moved relative to each other to set a desired space between the two pins. In other embodiments, instead of a linear slot, a circular, or generally, planar plate or substrate 468a may be used having radial slots (not shown) to couple with the pins 464a, 466a, and 478 in order to test the touch sensation in a two dimensional area rather than a straight line. The pins may be moved radially inwards towards the center or outwards towards the perimeter to decrease or increase the touch discrimination test area, respectively.

FIG. 4D shows an example removable diagnostic head configured to test touch sensitivity. In various embodiments, touch sensitivity diagnostic head 480 may include an attachment base 482, substrate 484 coupled with flexible filament 488 attached to filament base 486.

In various embodiments, touch sensitivity diagnostic head 480 is used to test how sensitive a patient's skin is to touch. For example, in diabetic patients the touch sensation in the extremities like fingers and toes is reduced. So, a touch sensitivity test helps physicians as an indicator to determine the extent or progress of the disease in the patient.

In various embodiments, filament 488 may be a Semmes-Weinstein™ monofilament configured to bend at a predetermined amount of exerted force, for example, 10 grams. The filament is similar to a short, on the order of one or a few inches, thick fishing line which is brought to contact with the patient's skin. The bending force is an indicator of how much force needs to be exerted on the patient's skin before he feels the touch. In various embodiments, the filament may be disposable or reusable. The filament may also be available in various predetermined force ratings. For example, a kit may include a number of filament “pens” or assemblies including filament base 486 coupled with the filament 488, each filament pen with a predetermined force rating, such as 5 grams, 10 grams, 15 grams, etc. In various embodiments, the filament pen may be pressure fit or screwed into a hole built into the substrate 484, as shown in dotted lines.

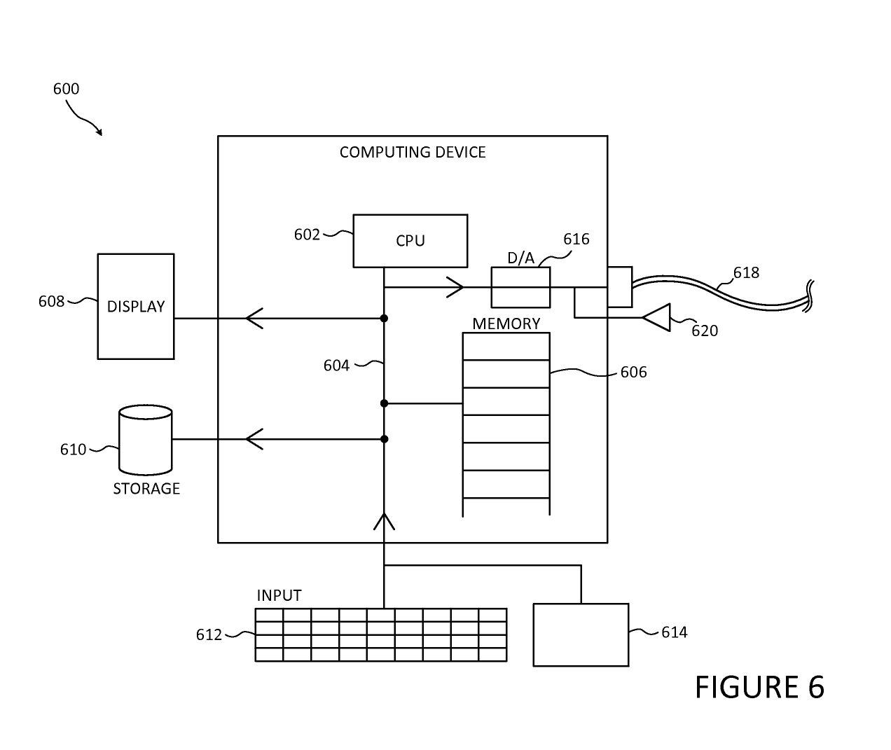

FIG. 5 shows an example removable diagnostic head configured to test hearing acuity using a computing device. In various embodiments, hearing test system 500 includes computing device 508 having touch screen 512, hardware buttons 516 and 518, software application generated waveforms 514, signal cable 510 connecting Otoscope 506 to wired hearing diagnostic head 502 to generate test sound signal 504. In some embodiments, a wireless hearing diagnostic head 520 having wireless interface and processing 522 may be used to receive wireless signal 524 transmitted from computing device 508.

In various embodiments, various aspects of the hearing of a patient, such as sound intensity and various frequencies and tones may be tested using various sound waves generated by a sound generation software application running on computing device 508. The sound generation software application may provide a user interface to display options for generating a sound signal to be used to test the patient's hearing. The options may include the sound intensity, wave pattern, sound frequency, and wave harmonics. In various embodiments, the physician or user of the medical device may dynamically change any of the options while the patient's hearing is being tested using the interface controls provided. Such dynamic or real-time changes may include change of sound intensity (wave amplitude), frequency, wave patterns, and the like. In various embodiments, the medical device user may generate a fixed pitch, such as a fixed frequency sinusoidal wave or a variable pitch using a pre-determined wave pattern such as a square wave having many frequency harmonics.

In various embodiments, the computing device may be a portable device such as a smartphone, a tablet computer, a music player, or a computing pad with a sound generation app (small software application for mobile devices). The computing device may also be a laptop computer, a desktop computer, or a remote server. In various embodiments, sound generation software application may run on a device locally, or it may be transmitted from a remote computer, such as central server in a hospital or a clinic.

In various embodiments, hearing diagnostic head 502 may have a body or attachment base configured to be coupled with the otoscope and a tip for producing sound. The hearing diagnostic head may have a wired interface to connect to signal cable 510 for receiving the sound signal. The other end of the signal cable may connected to any source of sound signal such as a computing device, a music player, an MP3 or MP4 player, and the like.

In other various embodiments, the hearing diagnostic head may have a wired interface like USB (Universal Serial Bus), or a wireless interface, such as Bluetooth, WiFi, or other wireless interface which receives the audio signal from the computing device having a similar or compatible interface for transmission of wired or wireless signals. In various embodiments, a small processing module may be embedded in or integrated with the hearing diagnostic head having a wireless interface and other processing capability, such as signal filtering and amplification, as needed to transform the wireless signal into audio signal. In various embodiments, the integrated processing module may be implemented in hardware, in software, or a combination thereof.

In various embodiments, the sound signal 504 may be generated as a wave pattern with many harmonics, or a single frequency wave or a simple oscillating function like a sinusoid. The sound signal may also be pre-generated and saved in an audio file for playback. Such audio files may be easily copied, transferred, played back, transmitted, or processed in the same ways a music file can.

In various embodiments, the computing device may also output pre-defined commands to other diagnostic heads to output certain sensory stimuli at certain appropriate levels, such as heat, cold, force, electrical current, and the like. For example, if an electric shock diagnostic head is being used, the user may control the voltage and/or current level via the computing device. Similarly, the user may control the temperature output from a thermal diagnostic head via commands from the computing device.

In various embodiments, the type and value of the sensory stimulus applied by the medical diagnostic device is recorded in the memory of the external computing device, or the memory of the integrated computing device within the diagnostic head or the medical diagnostic device. Such information may then be transmitted, by wire or wirelessly, to a recording device external to the medical diagnostic device, such as a computer or remote server.

In various embodiments, the sensory stimuli, wave patterns, timing, frequency, temperature, or other stimuli

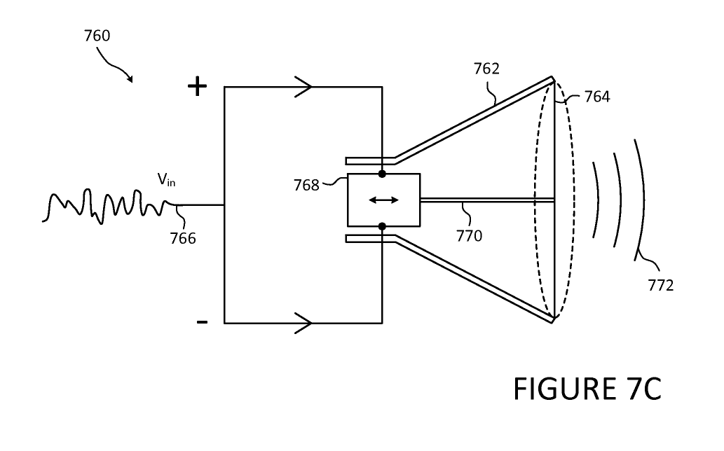

In various embodiments, the hearing diagnostic head 502 may be adapted for diagnosing the sense of touch. As further described below with respect to FIGS. 7A-7C, the hearing diagnostic head may include one of a variety of loudspeakers to generate sound. The amplitude of the generated sound wave may be intense enough to be sensed as vibrations by the sense of touch in skin. As such, this technique may be used to for deep tissue sensory diagnostics. For example, the diagnostic head so equipped may be placed on the skin and be used to transmit various frequencies and intensities to test the patient's ability to sense and/or differentiate different frequencies and intensities of vibration on various points on his skin.

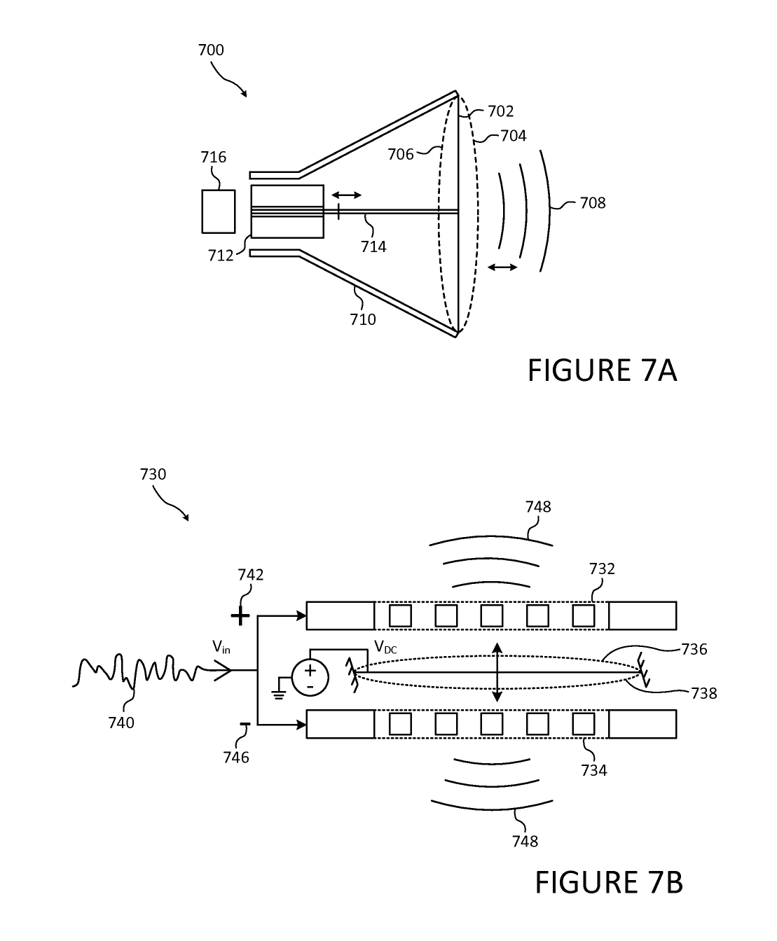

FIG. 6 shows an example computing device similar to the computing device of FIG. 5. In various embodiments, the computing device 600 may include CPU (Central Processing Unit) 602, signal bus 604, memory 606, display output 608, non-volatile storage 610, input devices such as keyboard 612 and touch pad 614, D/A (Digital to Analog converter) 616 coupled with antenna 620 and signal cable 618.

In various embodiments, any general computing device such as a Personal Computer (PC) of various sorts like laptops, desktops, and tablets may be used as a computing device for diagnostic and sound generation and/or playback purposes. Those skilled in the art will appreciate that the computing device 600 may have fewer or more components than shown. Memory 606 may include volatile RAM (Random Access Memory) and non-volatile ROM (Read Only Memory), and is generally used to load and execute programs and data during execution and/or boot-up of the computing device. The sound generation software may be one of the applications that is loaded into the memory. The D/A may be used by the CPU to convert digital data generated by the sound generation software application into an analog signal which may then be transmitted via a cable and/or by wireless antenna 620 to the hearing diagnostic head 502, shown in FIG. 5, to create a sound.

Those skilled in the art will appreciate that the sound generation software application may include one or more software modules to generate the sound, display user options and controls, retrieve sound files, playback sound files and the like. In various embodiments, particular wave pattern selected and/or configured by the user to be generated by the sound generator may be stored as a file for later use. This way, once a user configures a useful wave pattern for a particular type of hearing test or diagnostic can store the wave pattern as a sound file for future use instead of trying to reconfigure the wave pattern every time. Such audio files may be used for a particular patient as a personalized test, or it may be used for a particular type of hearing test for all patients.

In various embodiments, hearing diagnostic head 502 may include any form of loudspeaker that is small enough to fit on a otoscope and precise enough to make sound waves with sufficiently low distortion and noise suitable for performing a medical hearing test. A number of such loudspeakers are described below with respect to FIGS. 7A-7C.

In various embodiments, the computing device may be external with respect to the diagnostic head, as described above, or internal. The computing device may include a small microcontroller with integrated storage and program memory deployed within the diagnostic head as one integrated unit. In such embodiments, the user may have a wireless or wired interface configured to issue simple commands for changing the frequency, amplitude, and wave patterns. In other embodiments, the diagnostic head may be preprogrammed for a fixed wave pattern and amplitude. In still other embodiments, the wave pattern may be fixed and the intensity may be adjusted or varied via a slider or dial on the diagnostic head. In such embodiments, a set of multiple heads may be used, each one preprogrammed to generate a particular wave pattern and/or intensity.

FIG. 7A shows an example cone loudspeaker. In various embodiments, the hearing diagnostic head 502 shown in FIG. 5, may include cone loud speaker 700, which may include a cone-shaped body or frame 710, electromagnet 712, permanent magnet 716, diaphragm 702 coupled to connecting member 714 and configured to vibrate between positions 704 and 706 to generate sound 708, with the connecting member 714 being coupled with and driven by electromagnet 712.

In various embodiments, electromagnet 712 is coupled with an electrical signal representing sound wave patterns. When the electrical signal changes, the electromagnet, in conjunction with the permanent magnet 716, move the connecting member 714 and cause the diaphragm 702 to vibrate and create the sound 708 embedded in the electrical signal.

Cone loudspeaker 700 is a common device used in many audio equipment and devices.

A variation of the cone speaker is vibration loudspeaker, which is similar in construction and function to the cone speaker, except that there is no diaphragm. Instead, the diaphragm is replaced with a moveable rigid plate, which, when attached to a surface causes the surface to vibrate accordingly and act as a diaphragm which in turn moves the air and makes the sound. In effect, the surface to which the vibration speaker is attached behaves like the diaphragm of the cone speaker and makes the sound. This way, the vibration speaker can be used as a small and portable speaker which can be attached to many surfaces, such as a window, a table, a wall, a panel, or other similar surfaces to produce a rich sound.

The vibration speaker may be substantially used in place of the cone speaker in this disclosure.

FIG. 7B shows an example graphene loudspeaker. In various embodiments, loudspeaker 730 may include perforated electrodes 732 and 734 to conduct non-inverting input signal 742 and inverted input signal 746, both sourced from input signal 740, and allow generated sound 748 to be emitted from the perforations. A graphene diaphragm 736, made of very light weight and strong carbon mesh, biased with a DC (Direct Current) voltage and configured to vibrate as signified by dotted lines 738.

In various embodiments, the low mass and strong graphene diaphragm vibrates in the electromagnetic field generated by the input signal 740 through electrodes 732 and 734. Desirable frequency response characteristics, such as constant pressure at human hearing frequency ranges of about 20 Hertz to 20,000 (20 K) Hertz and low dampening requirements, make this type of loudspeaker suitable for applications in which small and size and light weight is desirable, such as medical diagnostic applications.

FIG. 7C shows an example piezoelectric loud speaker. In various embodiments, piezoelectric loudspeaker 760 may include frame 762, diaphragm 764 coupled with piezoelectric Chrystal 768 via connecting member 770 and configured to be coupled with input signal 766 to generate sound waves 772.

In various embodiments, piezoelectric Chrystal 768 is configured to expand and contract in response to an applied voltage in the direction of the arrow shown. A variable input signal causes the Chrystal to move in a vibrational pattern, thus causing a vibration in the diaphragm 764, as indicated by the dotted lines, to generate sound 772.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A medical diagnostic system comprising:
a handle;
a hearing diagnostic head including an attachment base and a loudspeaker configured to provide a sound wave; and
a computing device coupled with the hearing diagnostic head and configured to generate a signal.

2. The medical diagnostic system of claim 1, wherein the hearing diagnostic head includes a wireless interface.

3. The medical diagnostic system of claim 1, wherein the handle is powered by one of a battery and a power cord.

4. The medical diagnostic system of claim 1, wherein the hearing diagnostic head is removable.

5. The medical diagnostic system of claim 1, wherein the hearing diagnostic head comprises one of a cone loudspeaker, a vibration loudspeaker, a graphene loudspeaker, and a piezoelectric loudspeaker.

6. The medical diagnostic system of claim 1, wherein the computing device is one of a smartphone, a computing pad, a computing tablet, and a music player.

7. The medical diagnostic system of claim 1, wherein the hearing diagnostic head is configured to generate a sound based on the signal generated by the computing device.

8. The medical diagnostic system of claim 1, wherein the signal generated by the computing device is selected by a user of the computing device.

9. A medical diagnostic system comprising:
a powered handle;
a hearing diagnostic head including an attachment base and a diagnostic tip, wherein the diagnostic tip is configured to generate a sound wave; and
a computing device coupled with the hearing diagnostic head, configured to generate a signal based on which the sound wave is generated by the diagnostic tip.

10. The medical diagnostic system of claim 9, wherein the powered handle is powered by battery or by an electrical cord.

11. The medical diagnostic system of claim 9, wherein the hearing diagnostic head is removable.

12. The medical diagnostic system of claim 9, wherein the hearing diagnostic tip comprises a loudspeaker.

13. The medical diagnostic device of claim 12, wherein the loudspeaker is one of a cone loudspeaker, a vibration loudspeaker, a graphene loudspeaker, and a piezoelectric loudspeaker.

14. The medical diagnostic system of claim 9, wherein the signal generated by the computing device is generated by a software application module running on the computing device.

15. The medical diagnostic system of claim 9, wherein the signal generated by the computing device is pre-recorded.

16. A method of testing hearing, the method comprising:
using a medical diagnostic system including a computing device coupled with a hearing diagnostic head;
selecting a wave pattern based on which a signal is provided by the computing device;
transmitting the signal to the hearing diagnostic head; and
producing a sound by the hearing diagnostic head based on the transmitted signal.

17. The method of claim 16, wherein the hearing diagnostic head is configured to be coupled to an otoscope.

18. The method of claim 16, wherein the hearing diagnostic head includes one of a cone loudspeaker, a vibration loudspeaker, a graphene loudspeaker, and a piezoelectric loudspeaker.

19. The method of claim 16, wherein the selected wave pattern is generated by a software application module running on the computing device.

20. The method of claim 16, wherein the computing device is one of a smartphone, a computing pad, a computing tablet, and a music player.
